(19) United States
(12) Patent Application Publication
(10) Pub. No.: US 2001/0014330 A1
(43) Pub. Date: Aug. 16, 2001

(54) IMMUNIZATION AGAINST ENDOGENOUS MOLECULES

(76) Inventors: RICHARD HARLAND, SASKATOON (CA); JOHN G. MANNS, SASKATOON (CA); STEPHEN D. ACRES, SASKATOON (CA)

Correspondence Address: ROBINS & PASTERNAK LLP, 90 MIDDLEFIELD ROAD, SUITE 200, MENLO PARK, CA, 94025, US

(21) Appl. No.: 09/019,010

(22) Filed: Feb. 5, 1998

Related U.S. Application Data
(60) Provisional application No. 60/036,883, filed on Feb. 5, 1997.

(51) Int. Cl. A61K 39/00; A61K 39/38

(52) U.S. Cl. 424/184.1

(57) ABSTRACT

A method is described for immunoneutralization of endogenous molecules in mammalian subjects, wherein an immunogen is administered via injection to the ear. The method is used to elicit an efficient and uniform immune response sufficient to block or suppress the activity of an endogenous hormone in a vaccinated subject, or to target a diseased cell for an immune response.

### CROSS-REFERENCE TO RELATED APPLICATION

This application is related to provisional patent application serial no. 60/036,883, filed Feb. 5, 1997, from which priority is claimed under 35 U.S.C. §119 (e) (1) and which is incorporated herein by reference in its entirety.

### TECHNICAL FIELD

The present invention relates generally to active immunization against endogenous molecules. More particularly, the invention relates to methods for immunoneutralization of endogenous molecules in mammalian subjects, wherein the immunogen is administered via injection to the ear.

### BACKGROUND OF THE INVENTION

A number of vaccination methods have been suggested for use in the control of fertility or reproductive function in mammals. These vaccines operate by eliciting an immune response against an endogenous hormone in the vaccinated subject which is effective to neutralize the activity of the hormone. For example, immunological methods have been used to elicit an immune response against the reproductive hormone human chorionic gonadotropin (Matsuura et al. (1979) Endocrinol. 101:396-401). Other targets include two gonadotrophic hormones known to be involved in the control of the estrus cycle, particularly luteinizing hormone (LH) and follicle stimulating hormone (FSH). In vertebrates, synthesis and release of these two hormones are regulated by a polypeptide referred to as Gonadotropin releasing hormone (GnRH) (formerly designated LHRH). Accordingly, an approach to fertility control in an animal population is to reduce the levels of GnRH, such as by immunization against endogenous GnRH, which effects a reduction in the levels of LH and FSH and the concomitant disruption of estrous cycles and spermatogenesis. See e.g., Adams et al. (1990) J. Anim. Sci. 68:2793-2802.

Early studies of the GnRH molecule have shown that it is possible to raise antisera in response to repeated injections of synthetic GnRH peptides (Arimura et al. (1973) Endocrinology 93(5):1092-1103). Further, antibodies to GnRH have been raised in a number of species by chemical conjugation of GnRH to a suitable carrier and administration of the conjugate in an appropriate adjuvant (Carelli et al. (1982) Proc. Natl. Acad. Sci. 79:5392-5395). Protein conjugates, and/or recombinant fusion proteins, comprising GnRH or GnRH analogues have also been described for use in peptide vaccines for the immunological castration or inhibition of reproductive function of various domesticated and farm animals (Meloen et al. (1994) Vaccine 12 (8):741-746; Hoskinson et al. (1990) Aust. J. Biotechnol. 4:166-170; and International Publication Nos. WO 96/24675, published Aug. 15, 1996, WO 92/19746, published Nov. 12, 1992; WO 91/02799, published Mar. 7, 1991; WO 90/11298, published Oct. 4, 1990 and WO 86/07383, published Dec. 18, 1986).

However, there remains a need for a method for vaccinating against these and other endogenous molecules, wherein the method provides for enhanced uniformity and efficacy in the immune response directed against the target molecule. There also remains a need for such a method which can be practiced safely in a field setting, thereby reducing the incidence of inappropriate or accidental administration of the vaccine to the person delivering the vaccine.

### DISCLOSURE OF THE INVENTION

The present invention is based on the discovery that vaccination against endogenous molecules can be carried out in a highly uniform and efficient manner by delivery of immunogens to a mammalian subject via injection to the ear.

In one embodiment, the invention pertains to a method for presenting a selected endogenous immunogen to a mammalian subject by administering to the subject's ear a vaccine composition containing the immunogen. Administration can be carried out using conventional needle and syringe devices, needleless delivery devices or, preferably, using a jet injector device.

In another embodiment, the invention is directed to a method for inducing a uniform immune response against an endogenous hormone in a mammalian subject by administering to the ear of the subject a vaccine composition containing an immunogen derived from the hormone. The vaccine composition is capable of inducing an immune response against the subject endogenous hormone.

In yet another embodiment, the invention is directed to a method for inducing a uniform immune response against an endogenous hormone receptor in a mammalian subject by administering to the ear of the subject a vaccine composition containing an immunogen derived from the hormone receptor. The vaccine composition is capable of inducing an immune response against the subject endogenous hormone receptor, thereby neutralizing the biological activity, e.g., ligand binding activity, of that molecule.

Thus, in one aspect of the invention, methods are provided for immunoneutralization of endogenous hormones and/or hormone receptors by vaccines that are delivered to the ear. The vaccines contain an endogenous immunogen derived from the target molecule, either alone, or in combination with a suitable carrier molecule, and are injected either subcutaneously, subdermally, or intradermally into the pinna of the external ear.

In one particular embodiment, the invention entails delivery of a selected GnRH immunogen to a mammalian subject to immunocastrate the vaccinated animal.

The methods can be practiced in any suitable mammalian subject, however, commercially significant domestic animals are especially contemplated. For example, the methods of the present invention can be practiced in porcine subjects to reduce boar taint, or as an alternative to surgical castration in cattle.

These and other embodiments of the present invention will readily occur to those of ordinary skill in the art in view of the disclosure herein.

### BRIEF DESCRIPTION OF THE FIGURES

FIGS. 1A and 1B show the nucleotide sequences and amino acid sequences of the GnRH constructs used in the chimeric leukotoxin-GnRH polypeptide gene fusions. FIG. 1A depicts GnRH-1 which includes a single copy of a GnRH decapeptide; FIG. 1B depicts GnRH-2 which includes four copies of a GnRH decapeptide when n=1, and eight copies of GnRH when n=2, etc.

### DETAILED DESCRIPTION

The practice of the present invention will employ, unless otherwise indicated, conventional techniques of molecular biology, microbiology, virology, recombinant DNA technology, and immunology, which are within the skill of the art. Such techniques are explained fully in the literature. See, e.g., Sambrook, Fritsch & Maniatis, Molecular Cloning: A Laboratory Manual; DNA Cloning, Vols. I and II (D. N. Glover ed.); Oligonucleotide Synthesis (M. J. Gait ed.); Nucleic Acid Hybridization (B. D. Hames & S. J. Higgins eds.); Animal Cell Culture (R. K. Freshney ed.); Immobilized Cells and Enzymes (IRL press); B. Perbal, A Practical Guide to Molecular Cloning; the series, Methods In Enzymology (S. Colowick and N. Kaplan eds., Academic Press, Inc.); and Handbook of Experimental Immunology, Vols. I-IV (D. M. Weir and C. C. Blackwell eds., Blackwell Scientific Publications).

All patents, patent applications, and publications mentioned herein, whether supra or infra, are hereby incorporated by reference in their entirety.

A. Definitions

In describing the present invention, the following terms will be empolyed, and are intended to be defined as indicated below.

An “immunogen” refers to any agent, generally a macromolecule, which can elicit an immunological response in an individual. The immunological response may be of B- and/or T-lymphocytic cells. The term may be used to refer to an individual macromolecule or to a homogeneous or heterogeneous population of antigenic macromolecules.

An “immunological response” to an immunogen or vaccine is the development in the host of a cellular and/or antibody-mediated immune response to the immunogen or vaccine of interest. Usually, such a response includes but is not limited to one or more of the following effects; the production of antibodies, B cells, helper T cells, suppressor T cells, and/or cytotoxic T cells and/or γδ cells, directed specifically to an immunogen or immunogens included in a composition or vaccine of interest. An immunological response can be detected using any of several immunoassays well known in the art.

The phase “endogenous immunogen,” as used herein, refers to all, or a portion, of a targeted endogenous cellular component against which an immune response is to be raised. The term thus includes molecules (immunogens) derived from peptide and steroid hormones, hormone receptors, hormone agonists, hormone antagonists; cancer-associated markers and/or antigens; and the like, which molecules are capable of being rendered immunogenic, or more immunogenic, by way of association with a carrier molecule, by mutation of a native sequence, and/or by incorporation into a multimer containing multiple repeating units of at least an epitope of a subject endogenous immunogen. The term includes peptide molecules having amino acid substitutions, deletions and/or additions and which have at least about 50% amino acid identity to the reference molecule, more preferably about 75-85% identity and most preferably about 90-95% identity or more, to the relevant portion of the native peptide sequence in question. Expressly excluded from the definition of “endogenous immunogen” are any moieties derived from an infectious agent such as a bacterium or a virus.

An “epitope” refers to any portion or region of a molecule with the ability or potential to elicit, and combine with, specific antibody. For the purpose of the present invention, a polypeptide epitope will usually include at least about 3 amino acids, preferably at least about 5 amino acids, more preferably at least about 10-15 amino acids, and most preferably 25 or more amino acids, of the reference molecule. There is no critical upper limit to the length of the fragment, which could comprise nearly the full-length of a protein sequence, or even a fusion protein comprising two or more epitopes of a protein in question. Epitopes in polypeptide molecules can be identified using any number of eptiope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear eptiopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. U.S.A. 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra.

By “carrier” is meant any molecule which, when associated with an endogenous immunogen of interest, imparts immunogenicity to that molecule. Examples of suitable carriers include large, slowly metabolized macromolecules such as: proteins; polysaccharides, such as sepharose, agarose, cellulose, cellulose beads and the like; polymeric amino acids such as polyglutamic acid, polylysine, and the like; amino acid copolymers; inactive virus particles; bacterial toxins such as tetanus toxoid, leukotoxin molecules, and the like. Carriers are described in further detail below.

An endogenous immunogen is “linked” to a specified carrier molecule when the immunogen is chemically coupled to the carrier, or when the immunogen is expressed from a chimeric DNA molecule which encodes the immunogen and the carrier of interest.

“Native” proteins or polypeptides refer to proteins or polypeptides isolated from the source in which the proteins naturally occur. “Recombinant” polypeptides refer to polypeptides produced by recombinant DNA techniques; i.e., produced from cells transformed by an exogenous DNA construct encoding the desired polypeptide. “Synthetic” polypeptides are those prepared by chemical synthesis.

A “vector” is a replicon, such as a plasmid, phage, or cosmid, to which another DNA segment may be attached so as to bring about the replication of the attached segment.

A DNA “coding sequence” or a “nucleotide sequence encoding” a particular protein, is a DNA sequence which is transcribed and translated into a polypeptide in vitro or in vivo when placed under the control of appropriate regulatory elements. The boundaries of the coding sequence are determined by a start codon at the 5′ (amino) terminus and a translation stop codon at the 3′ (carboxy) terminus. A coding sequence can include, but is not limited to, procaryotic sequences, cDNA from eucaryotic mRNA, genomic DNA sequences from eucaryotic (e.g., mammalian) DNA, and even synthetic DNA sequences. A transcription termination sequence will usually be located 3′ to the coding sequence.

The term DNA “control elements” refers collectively to promoters, ribosome binding sites, polyadenylation signals, transcription termination sequences, upstream regulatory domains, enhancers, and the like, which collectively provide for the transcription and translation of a coding sequence in a host cell. Not all of these control sequences need always be present in a recombinant vector so long as the desired gene is capable of being transcribed and translated.

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

A control element, such as a promoter, “directs the transcription” of a coding sequence in a cell when RNA polymerase will bind the promoter and transcribe the coding sequence into mRNA, which is then translated into the polypeptide encoded by the coding sequence.

A “host cell” is a cell which has been transformed, or is capable of transformation, by an exogenous nucleic acid molecule.

A cell has been “transformed” by exogenous DNA when such exogenous DNA has been introduced inside the cell membrane. Exogenous DNA may or may not be integrated (covalently linked) into chromosomal DNA making up the genome of the cell. In procaryotes and yeasts, for example, the exogenous DNA may be maintained on an episomal element, such as a plasmid. With respect to eucaryotic cells, a stably transformed cell is one in which the exogenous DNA has become integrated into the chromosome so that it is inherited by daughter cells through chromosome replication. This stability is demonstrated by the ability of the eucaryotic cell to establish cell lines or clones comprised of a population of daughter cells containing the exogenous DNA.

The term “derived from,” as it is used herein, denotes an actual or theoretical source or origin of the subject molecule or immunogen. For example, an immunogen that is “derived from” a particular hormone molecule will bear close sequence similarity with a relevant portion of the hormone. Thus, an immunogen that is “derived from” a GnRH hormone may include all of the wild-type GnRH sequence, or may be altered by insertion, deletion or substitution of amino acid residues, so long as the derived sequence provides for an immunogen that corresponds to the targeted hormone. Immunogens derived from a denoted molecule will contain at least one epitope specific to the denoted molecule.

By “mammalian subject” is meant any member of the class mammalia, including, without limitation, rodents, cattle, pigs, sheep, goats, horses and primates and companion animals such as dogs and cats. The term does not denote a particular age. Thus, adults, newborns, and fetuses are intended to be covered.

B. General Methods

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of compositions and methods similar or equivalent to those described herein can be used in the practice of the present invention, the preferred materials and methods are described herein.

Central to the instant invention is the discovery that the efficiency and, particularly, the uniformity, of vaccination against an endogenous immunogen can be greatly increased in mammalian subjects through the administration of vaccine compositions to the ear instead of intramuscular administration into the neck. In commercially significant domestic animals, the ear provides a desirable site for such injections since the ear is not generally consumed by humans. This avoids the presence of residual immunogens and/or other vaccine components (e.g., oils) in consumable tissue at time of slaughter, particularly when such residuals may have adverse affects on humans. In cattle, the ear is an ideal vaccination site since it is not consumed. In swine, the ear is also a preferred site for such vaccinations since it provides a readily accessible location for subcutaneous or intradermal injection.

Accordingly, one aspect of the invention relates to targeted delivery of vaccine compositions containing one or more endogenous immunogens. Delivery is carried out by administering the vaccine composition to a subject's ear. The vaccine compositions are used to induce production of antibodies capable of neutralizing the bioactivity of a targeted endogenous hormone, hormone receptor, agonist or antagonist; or are used to elicit an immune response against a targeted endogenous cell type (e.g., a cancerous or otherwise diseased cell). These “self” molecules must be rendered immunogenic in order to be recognized by a vaccinated subject's immune system. The vaccine compositions thus generally comprise one or more epitopes derived from an endogenous molecule, and are provided as nucleic acid- and/or peptide-based compositions.

The endogenous immunogen can be derived from peptide hormones, such as ACTH, CRF, GHRH, GnRH, cholecystokinin, dynorphins, endorphins, endothelin, fibronectin fragments, galanin, gastrin, insulin, proinsulin, growth hormone, EGF, Somatostatin, SNX-111, BNP, insulinotropin, glucagon, ANP, GTP-binding protein fragments, the leukokinins, magainin, mastoparans, dermaseptin, systemin, neuromedins, neurotensin, pancreastatin, pancreatic polypeptide, vasoactive intestinal polypeptide (VIP), substance P, secretin, thymosin, and the like. The immunogen can likewise be derived from a glycoprotein hormone (e.g., thryoid-stimulating hormone (TSH), follicle-stimulating hormone (FSH), luteinizing hormone (LH), placental hormones, and chorionic gonadotropin (hCG)), or a steroid hormone (e.g., gonadal steroid hormones such as androgens, estrogens and progesterone). Other endogenous immunogens can be derived from peptide hormone receptors (e.g., insulin receptor, angiotensin receptor, growth hormone receptor, and the like), or from any member of the superfamily of steroid hormone receptors. Immunogens derived from hormone agonists (activin) and antagonists (e.g., inhibin) also find use in the present vaccine compositions, as well as tumor antigens, for example, any of the various MAGEs (melanoma associated antigen E), including MAGE 1, 2, 3, 4, etc. (Boon, T. (1993) Scientific American pp 82-89); any of the various tyrosinases; MART 1 (melanoma antigen recognized by T cells), mutant ras; mutant p53; p97 melanoma antigen; CEA (carcinoembryonic antigen); and the like; or embryonic proteins that have been re-expressed by transformed cells, or autoantigens that are not truly tumor specific, but are prevalent or overexpressed in mammalian tumor tissue.

It is generally understood that the immunogenicity of endogenous molecules may be significantly increased by producing immunogenic forms of such molecules comprising multiple copies of selected epitopes. Accordingly, in one aspect of the invention, vaccine compositions containing endogenous immunogen multimers are provided in either nucleic acid or peptide form for targeted delivery to a subject's ear.

The endogenous immunogens may also be conjugated to a suitable carrier in order to elicit an immune response in a challenged host. Suitable carriers are generally polypeptides or proteins which include antigenic regions of a protein derived from an infectious material such as a viral surface protein, or a carrier peptide sequence. These carriers serve to non-specifically stimulate T-helper cell activity and to help direct an immunogen of interest to antigen presenting cells (APCs) for processing and presentation at the cell surface in association with molecules of the major histocompatibility complex (MHC).

Several carrier systems have been developed for this purpose. For example, small peptide haptens are often coupled to protein carriers such as keyhole limpet hemocyanin (Bittle et al. (1982) Nature 298:30-33), bacterial toxins such as tetanus toxoid (Muller et al. (1982) Proc. Natl. Acad. Sci. U.S.A. 79:569-573), ovalbumin, and sperm whale myoglobin, to produce an immune response. These coupling reactions typically result in the incorporation of several moles of peptide hapten per mole of carrier protein.

Other suitable carriers for use with the present invention include VP6 polypeptides of rotaviruses, or functional fragments thereof, as disclosed in U.S. Pat. No. 5,071,651. Also useful is a fusion product of a viral protein and one or more epitopes from a targeted molecule of interest, which fusion products are made by the methods disclosed in U.S. Pat. No. 4,722,840. Still other suitable carriers include cells, such as lymphocytes, since presentation in this form mimics the natural mode of presentation in the subject, which gives rise to the immunized state. Alternatively, the endogenous immunogens may be coupled to erythrocytes, preferably the subject's own erythrocytes. Methods of coupling peptides to proteins or cells are known to those of skill in the art.

Delivery systems useful in the practice of the present invention may also utilize particulate carriers. For example, pre-formed particles have been used as platforms onto which immunogens can be coupled and incorporated. Systems based on proteosomes (Lowell et al. (1988) Science 240:800-802) and immune stimulatory complexes (Morein et al. (1984) Nature 308:457-460) are also known in the art.

Carrier systems using recombinantly produced chimeric proteins that self-assemble into particles may also be used with the present invention. For example, the yeast retrotransposon, Ty, encodes a series of proteins that assemble into virus like particles (Ty-VLPs; Kingsman et al. (1988) Vaccines 6:304-306). Thus, a gene, or fragment thereof, encoding the endogenous immunogen of interest may be inserted into the TyA gene and expressed in yeast as a fusion protein. The fusion protein retains the capacity to self assemble into particles of uniform size. Other useful virus-like carrier systems are based on HBsAg, (Valenzuela et al. (1985) Bio/Technol. 3:323-326; U.S. Pat. No. 4,722,840; Delpeyroux et al. (1986) Science 233:472-475), Hepatitis B core antigen (Clarke et al. (1988) Vaccines 88 (Ed. H. Ginsberg, et al.) pp. 127-131), Poliovirus (Burke et al. (1988) Nature 332:81-82), and Tobacco Mosaic Virus (Haynes et al. (1986) Bio/Technol. 4:637-641).

Especially preferred carriers include serum albumins, keyhole limpet hemocyanin, ovalbumin, sperm whale myoglobin, leukotoxin molecules, and other proteins well known to those skilled in the art.

Protein carriers may be used in their native form or their functional group content may be modified by, for example, succinylation of lysine residues or reaction with Cys-thiolactone. A sulfhydryl group may also be incorporated into the carrier (or antigen) by, for example, reaction of amino functions with 2-iminothiolane or the N-hydroxysuccinimide ester of 3-(4-dithiopyridyl propionate. Suitable carriers may also be modified to incorporate spacer arms (such as hexamethylene diamine or other bifunctional molecules of similar size) for attachment of peptide immunogens.

Carriers can be physically conjugated to the endogenous immunogen of interest, using standard coupling reactions. Alternatively, chimeric molecules can be prepared recombinantly for use in the present invention, such as by fusing a gene encoding a suitable polypeptide carrier to one or more copies of a gene, or fragment thereof, encoding for a selected endogenous immunogen.

1. Nucleic Acids

Generally, nucleic acid-based vaccines for use with the present invention will include relevant regions encoding an endogenous immunogen, with suitable control sequences and, optionally, ancillary therapeutic nucleotide sequences. The nucleic acid molecules are prepared in the form of vectors which include the necessary elements to direct transcription and translation in a recipient cell.

In order to augment an immune response in an immunized subject, the nucleic acid molecules can be administered in conjunction with ancillary substances, such as pharmacological agents, adjuvants, cytokines, or in conjunction with delivery of vectors encoding biological response modifiers such as cytokines and the like.

Nucleotide sequences selected for use in the present invention can be derived from known sources, for example, by isolating the same from cells or tissue containing a desired gene or nucleotide sequence using standard techniques, or by using recombinant or synthetic techniques.

Once coding sequences for the endogenous immunogen have been prepared or isolated, such sequences can be cloned into any suitable vector or replicon. Numerous cloning vectors are known to those of skill in the art, and the selection of an appropriate cloning vector is a matter of choice. Ligations to other sequences, e.g., ancillary molecules or carrier molecules, are performed using standard procedures, known in the art. One or more endogenous immunogen portions of the chimera can be fused 5′ and/or 3′ to a desired ancillary sequence or carrier molecule. Alternatively, one or more endogenous immunogen portions may be located at sites internal to the carrier molecule, or such portions can be positioned at both terminal and internal locations in the chimera.

Alternatively, DNA sequences encoding the endogenous immunogens of interest, optionally linked to carrier molecules, can be prepared synthetically rather than cloned. The DNA sequences can be designed with appropriate codons for the particular sequence. The complete sequence of the immunogen is then assembled from overlapping oligonucleotides prepared by standard methods and assembled into a complete coding sequence. See, e.g., Edge (1981) Nature 292:756; Nambair et al. (1984) Science 223:1299; and Jay et al. (1984) J. Biol. Chem. 259:6311.

The coding sequence is then placed under the control of suitable control elements for expression in suitable host tissue in vivo. The choice of control elements will depend on the subject being treated and the type of preparation used. Thus, if the subject's endogenous transcription and translation machinery will be used to express the immunogens, control elements compatible with the particular subject will be utilized. In this regard, several promoters for use in mammalian systems are known in the art. For example, typical promoters for mammalian cell expression include the SV40 early promoter, a CMV promoter such as the CMV immediate early promoter, the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression.

Typically, transcription termination and polyadenylation sequences will also be present, located 3′ to the translation stop codon. Preferably, a sequence for optimization of initiation of translation, located 5′ to the coding sequence, is also present. Examples of transcription terminator/polyadenylation signals include those derived from SV40, as described in Sambrook et al., supra, as well as a bovine growth hormone terminator sequence. Introns, containing splice donor and acceptor sites, may also be designed into the constructs for use with the present invention.

Enhancer elements may also be used herein to increase expression levels of the constructs. Examples include the SV40 early gene enhancer (Dijkema et al. (1985) EMBO J. 4:761), the enhancer/promoter derived from the long terminal repeat (LTR) of the Rous Sarcoma Virus (Gorman et al. (1982) Proc. Natl. Acad. Sci. USA 79:6777) and elements derived from human CMV (Boshart et al. (1985) Cell 41:521), such as elements included in the CMV intron A sequence.

Once prepared, the nucleic acid vaccine compositions can be delivered to the ear of a subject using known methods. In this regard, various techniques for immunization with antigen-encoding DNAs have been described. U.S. Pat. No. 5,589,466 to Felgner et al.; Tang et al. (1992) Nature 358:152; Davis et al. (1993) Hum. Molec. Genet. 2:1847; Ulmer et al. (1993) Science 258:1745; Wang et al. (1993) Proc. Natl. Acad. Sci. USA 90:4156; Eisenbraun et al. (1993) DNA Cell Biol. 12:791; Fynan et al. (1993) Proc. Natl. Acad. Sci. USA 90:12476; Fuller et al. (1994) AIDS Res. Human Retrovir. 10:1433; and Raz et al. (1994) Proc. Natl. Acad. Sci. USA 91:9519. General methods for delivering nucleic acid molecules to cells can also be used, such as liposome-mediated gene transfer. See, e.g., Hazinski et al. (1991) Am. J. Respir. Cell Mol. Biol. 4:206-209; Brigham et al. (1989) Am. J. Med. Sci. 298:278-281; Canonico et al. (1991) Clin. Res. 39:219A; and Nabel et al. (1990) Science 249:1285-1288. Thus, the nucleic acid vaccine compositions can be delivered in either liquid or particulate form using a variety of known techniques.

2. Peptides

Peptide-based vaccine compositions can also be produced using a variety of methods known to those skilled in the art. In particular, endogenous immunogens can be isolated directly from native sources, using standard purification techniques. Alternatively, the immunogens can be recombinantly produced using the nucleic acid expression systems described above, and purified using known techniques. Peptide immunogens can also be synthesized, based on described amino acid sequences or amino acid sequences derived from the DNA sequence of a molecule of interest, using chemical polymer syntheses such as solid phase peptide synthesis. Such methods are known to those skilled in the art. See, e.g., J. M. Stewart and J. D. Young, Solid Phase Peptide Synthesis, 2nd Ed., Pierce Chemical Co., Rockford, Ill. (1984) and G. Barany and R. B. Merrifield, The Peptides: Analysis, Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Verlag, Berlin (1984) and E. Gross and J. Meienhofer, Eds., The Peptides: Analysis, Synthesis, Biology, supra, Vol. 1, for classical solution synthesis.

Peptide immunogens may also be produced by cloning the coding sequences therefor into any suitable expression vector or replicon. Numerous cloning vectors are known to those of skill in the art, and the selection of an appropriate cloning vector is a matter of choice. Examples of recombinant DNA vectors for cloning, and host cells which they can transform, include the bacteriophage lambda (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomoyces), YCp19 (Saccharomyces) and bovine papilloma virus (mammalian cells). See, generally, DNA Cloning: Vols. I & II, supra; T. Maniatis et al., supra; B. Perbal, supra.

The gene can be placed under the control of a promoter, ribosome binding site (for bacterial expression) and, optionally, an operator, so that the DNA sequence of interest is transcribed into RNA by a suitable transformant. The coding sequence may or may not contain a signal peptide or leader sequence. The peptide immunogens can be expressed using, for example, the E. coli tac promoter or the protein A gene (spa) promoter and signal sequence. Leader sequences can be removed by the bacterial host in post-translational processing. See, e.g., U.S. Pat. Nos. 4,431,739; 4,425,437; 4,338,397.

In addition to control sequences, it may be desirable to add regulatory sequences which allow for regulation of the expression of the immunogen sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

An expression vector is constructed so that the particular coding sequence is located in the vector with the appropriate regulatory sequences, the positioning and orientation of the coding sequence with respect to the control sequences being such that the coding sequence is transcribed under the “control” of the control sequences (i.e., RNA polymerase which binds to the DNA molecule at the control sequences transcribes the coding sequence). Modification of the sequences encoding the particular endogenous immunogen may be desirable to achieve this end. For example, in some cases it may be necessary to modify the sequence so that it can be attached to the control sequences in the appropriate orientation; i.e., to maintain the reading frame. The control sequences and other regulatory sequences may be ligated to the coding sequence prior to insertion into a vector, such as the cloning vectors described above. Alternatively, the coding sequence can be cloned directly into an expression vector which already contains the control sequences and an appropriate restriction site.

In some cases, it may be desirable to add sequences which cause the secretion of the immunogen from the host organism, with subsequent cleavage of the secretory signal. It may also be desirable to produce mutants or analogues of the endogenous immunogen. Mutants or analogues may be prepared by the deletion of a portion of the sequence encoding the immunogen, or if present, a portion of the sequence encoding the desired carrier molecule, by insertion of a sequence, and/or by substitution of one or more nucleotides within the sequence. Techniques for modifying nucleotide sequences, such as site-directed mutagenesis, are well known to those skilled in the art. See, e.g., Sambrook et al., supra; DNA Cloning, Vols. I and II, supra; Nucleic Acid Hybridization, supra.

The endogenous immunogens can be expressed in a wide variety of systems, including insect, mammalian, bacterial, viral and yeast expression systems, all well known in the art. For example, insect cell expression systems, such as baculovirus systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit). Similarly, bacterial and mammalian cell expression systems are well known in the art and described in, e.g., Sambrook et al., supra. Yeast expression systems are also known in the art and described in, e.g., Yeast Genetic Engineering (Barr et al., eds., 1989) Butterworths, London.

A number of appropriate host cells for use with the above systems are also known. For example, mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Madin-Darby bovine kidney (“MDBK”) cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the endogenous immunogens are produced by growing host cells transformed by an expression vector described above under conditions whereby the immunogen is expressed. The expressed immunogen is then isolated from the host cells and purified. If the expression system secretes the immunogen into growth media, the product can be purified directly from the media. If it is not secreted, it can be isolated from cell lysates. The selection of the appropriate growth conditions and recovery methods are within the skill of the art.

Subjects can be immunized against endogenous immunogens by administration of vaccine compositions which include the above-described peptides. Prior to immunization, it may be desirable to further increase the immunogenicity of a particular immunogen. This can be accomplished in any one of several ways known to those of skill in the art. For example, the immunogen may be administered linked to a secondary carrier. Such carriers are described in detail above.

The immunogens can also be administered via a carrier virus which expresses the same. Carrier viruses which will find use herein include, but are not limited to, the vaccinia and other pox viruses, adenovirus, and herpes virus. By way of example, vaccinia virus recombinants expressing the proteins can be constructed as follows. The DNA encoding a particular protein is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the desired immunogen into the viral genome. The resulting TK-recombinant can be selected by culturing the cells in the presence of 5-bromodeoxyuridine and picking viral plaques resistant thereto.

Typically, the mammalian subject is immunized in the ear with the endogenous immunogen, either administered alone, or mixed with a pharmaceutically acceptable vehicle or excipient. Suitable vehicles are, for example, water, saline, dextrose, glycerol, ethanol, or the like, and combinations thereof. In addition, if desired, the vehicle may contain minor amounts of auxiliary substances such as wetting or pH buffering agents.

The vaccines are normally prepared as injectables, either as liquid solutions or suspensions, or as solid forms which are suitable for solution or suspension in liquid vehicles prior to injection. The preparation may also be emulsified or the active ingredient encapsulated in liposome vehicles. The active immunogenic ingredient is often mixed with vehicles containing excipients which are pharmaceutically acceptable and compatible with the active ingredient. Suitable vehicles are, for example, water, saline, dextrose, glycerol, ethanol, or the like, and combinations thereof. In addition, the vehicle may contain minor amounts of auxiliary substances such as wetting or emulsifying agents, pH buffering agents, or adjuvants which enhance the effectiveness of the vaccine. Suitable adjuvants include, for example, muramyl dipeptides, avridine, aluminum hydroxide, oils, saponins and other substances known in the art. Actual methods of preparing such dosage forms are known, or will be apparent, to those skilled in the art. See, e.g., Remington's Pharmaceutical Sciences, Mack Publishing Company, Easton, Pa., 18th edition, 1990. The composition or formulation to be administered will contain a quantity of the endogenous immunogen adequate to achieve the desired immunized state in the subject being treated.

Controlled or sustained release formulations are made by incorporating the endogenous immunogens into carriers or vehicles such as liposomes, nonresorbable impermeable polymers such as ethylenevinyl acetate copolymers and Hytrel® copolymers, swellable polymers such as hydrogels, or resorbable polymers such as collagen and certain polyacids or polyesters such as those used to make resorbable sutures.

The vaccine compositions may also be prepared in solid form for delivery to a subject's ear. For example, solid particulate formulations can be prepared for delivery from commercially available needleless injector devices. Alternatively, solid dose implants can be provided for implantation into a subject's ear, for example, using a trocar. See, e.g., Spitzer et al. (1978) Theriogenology 10:181-200; and Bretzlaff et al. (1991) Am. J. Vet. Res. 52:1423-1426.

Furthermore, the immunogens may be formulated into vaccine compositions in either neutral or salt forms. Pharmaceutically acceptable salts include the acid addition salts (formed with the free amino groups of the active polypeptides) and which are formed with inorganic acids such as, for example, hydrochloric or phosphoric acids, or organic acids such as acetic, oxalic, tartaric, mandelic, and the like. Salts formed from free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Other vaccine compositions can include adjuvants to further increase the immunogenicity of the endogenous immunogen. Adjuvants may include for example, emulsifiers, muramyl dipeptides, avridine, aluminum hydroxide, oils, saponins and other substances known in the art. More particularly, emulsifiers can be used as adjuvants. Compounds which may serve as emulsifiers herein include natural and synthetic emulsifying agents, as well as anionic, cationic and nonionic such compounds. Among the synthetic compounds, anionic emulsifying agents include, for example, the potassium, sodium and ammonium salts of lauric and oleic acid, the calcium, magnesium and aluminum salts of fatty acids (i.e., metallic soaps), and organic sulfonates such as sodium lauryl sulfate. Synthetic cationic agents include, for example, cetyltrimethylammonium bromide, while synthetic nonionic agents are exemplified by glyceryl esters (e.g., glyceryl monostearate), polyoxyethylene glycol esters and ethers, and the sorbitan fatty acid esters (e.g., sorbitan monopalmitate) and their polyoxyethylene derivatives (e.g., polyoxyethylene sorbitan monopalmitate). Natural emulsifying agents include acacia, gelatin, lecithin and cholesterol.

Other suitable adjuvants can be formed with an oil component, such as a single oil, a mixture of oils, a water-in-oil emulsion, or an oil-in-water emulsion. The oil may be a mineral oil, a vegetable oil, or an animal oil. Mineral oil, or oil-in-water emulsions in which the oil component is mineral oil are preferred. In this regard, a “mineral oil” is defined herein as a mixture of liquid hydrocarbons obtained from petrolatum via a distillation technique; the term is synonymous with “liquid paraffin,” “liquid petrolatum” and “white mineral oil.” The term is also intended to include “light mineral oil,” i.e., an oil which is similarly obtained by distillation of petrolatum, but which has a slightly lower specific gravity than white mineral oil. See, e.g., Remington's Pharmaceutical Sciences, supra, at pages 788 and 1323. A particularly preferred oil component is the oil-in-water emulsion sold under the trade name of EMULSIGEN PLUS™ (comprising a light mineral oil as well as 0.05% formalin, and 30 mcg/mL gentamicin as preservatives), available from MVP Laboratories, Ralston, Nebr., or the VSA-3 adjuvant which is a modified form of the EMULSIGEN PLUS™ adjuvant. Suitable animal oils include, for example, cod liver oil, halibut oil, menhaden oil, orange roughy oil and shark liver oil, all of which are available commercially. Suitable vegetable oils, include, without limitation, canola oil, almond oil, cottonseed oil, corn oil, olive oil, peanut oil, safflower oil, sesame oil, soybean oil, and the like.

Alternatively, a number of aliphatic nitrogenous bases can be used as adjuvants with the vaccine formulations. For example, known immunologic adjuvants include amines, quaternary ammonium compounds, guanidines, benzamidines and thiouroniums (Gall, D. (1966) Immunology 11:369-386). Specific such compounds include dimethyldioctadecylammonium bromide (DDA) (available from Kodak) and N,N-dioctadecyl-N,N-bis(2-hydroxyethyl)propanediamine (“avridine”). The use of DDA as an immunologic adjuvant has been described; see, e.g., the Kodak Laboratory Chemicals Bulletin 56(1):1-5 (1986); Adv. Drug Deliv. Rev. 5(3):163-187 (1990); J. Controlled Release 7:123-132 (1988); Clin. Exp. Immunol. 78(2):256-262 (1989); J. Immunol. Methods 97(2):159-164 (1987); Immunology 58(2):245-250 (1986); and Int. Arch. Allergy Appl. Immunol. 68(3):201-208 (1982). Avridine is also well-known as an adjuvant. See, e.g., U.S. Pat. No. 4,310,550 to Wolff, III et al., which describes the use of N,N-higher alkyl-N′,N′-bis(2-hydroxyethyl)propane diamines in general, and avridine in particular, as vaccine adjuvants. U.S. Pat. No. 5,151,267 to Babiuk, and Babiuk et al. (1986) Virology 159:57-66, also relate to the use of avridine as a vaccine adjuvant.

The vaccine composition is formulated to contain an effective amount of the endogenous immunogen, the exact amount being readily determined by one skilled in the art, wherein the amount depends on the animal to be treated, the capacity of the animal's immune system to synthesize antibodies, and the degree of protection desired. For peptide-based vaccine formulations, approximately 1 μg to 1 mg, more generally 5 μg to 200 μg of immunogen per mL of injected solution, should be adequate to raise an immunological response when administered. If a peptide-carrier chimera is used, the ratio of immunogen to carrier in the vaccine formulation will vary based on the particular carrier and immunogen selected to construct such molecules. Effective dosages can be readily established by one of ordinary skill in the art through routine trials establishing dose response curves. The subject is immunized by administration of one of the above-described vaccine compositions to the ear in at least one dose, and preferably two doses. Moreover, the animal may be administered as many doses as is required to maintain a state of immunity.

Any suitable pharmaceutical delivery means may be employed to deliver the vaccine composition to the subject's ear. For example, conventional needle syringes, spring or compressed gas (air) injectors (U.S. Pat. Nos. 1,605,763 to Smoot; 3,788,315 to Laurens; 3,853,125 to Clark et al.; 4,596,556 to Morrow et al.; and 5,062,830 to Dunlap), liquid jet injectors (U.S. Pat. Nos. 2,754,818 to Scherer; 3,330,276 to Gordon; and 4,518,385 to Lindmayer et al.), and particle injectors (U.S. Pat. Nos. 5,149,655 to McCabe et al. and 5,204,253 to Sanford et al.) are all appropriate for delivery of the vaccine compositions.

Preferably, the vaccine composition is administered subcutaneously, subdermally, or intradermally, to the subject's ear, for example, the pinna of the external ear. If a jet injector is used, a single jet of the liquid vaccine composition is ejected under high pressure and velocity, e.g., 1200-1400 PSI, thereby creating an opening in the skin and penetrating to depths suitable for immunization. When particularly small volumes of the vaccine are to be delivered by jet injection, for example, amounts less than about 0.1 mL, it may be more effective to deliver the vaccine to the hairless dorsal surface of the ear to avoid adverse effects of body hair.

Below are examples of specific embodiments for carrying out the present invention. The examples are offered for illustrative purposes only, and are not intended to limit the scope of the present invention in any way.

C. Experimental

Although the invention is broadly applicable to vaccination against any endogenous immunogen in a mammalian subject, the invention is exemplified herein with particular reference to active immunization against GnRH. Immunization against GnRH can be used to reduce boar taint in commercial swine, or used as an alternative to surgical castration in cattle. Bonneau et al. (1995) Livestock Production 42. A number of GnRH immunogens, vaccine compositions containing those immunogens, and methods of immunoneutralization against endogenous GnRH in vaccinated subjects using the vaccine compositions, are described in commonly owned U.S. patent application Ser. No. 08/694,865, filed Aug. 9, 1996, and in International Publication No. WO 96/24675, published Aug. 15, 1996.

Thus, one embodiment of the invention pertains to the delivery of a GnRH immunogen to the ear of a mammalian subject to provide an immune response directed against endogenous GnRH. The particular immunogen used can comprise one or more GnRH polypeptides, and/or one or more GnRH multimers. The selected GnRH immunogens can be used in their native form, or modified to provide a more immunogenic form, for example, by succinylation of lysine residues or reaction with Cys-thiolactone.

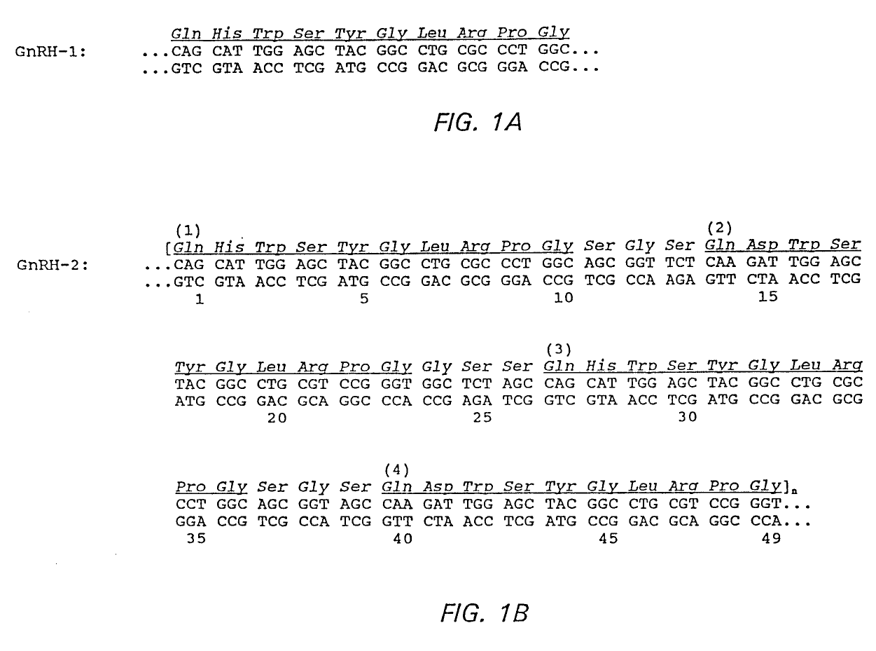

Further, the GnRH immunogen can be administered to the ear alone, or in combination with a suitable carrier molecule. Alternatively, the GnRH immunogen is conjugated to a macromolecular carrier, or a chimeric molecule can be used which includes leukotoxin fused to a GnRH polypeptide. More particularly, leukotoxin-GnRH chimeras are formed which include a leukotoxin polypeptide fused to one or more GnRH multimers having at least one repeating GnRH decapeptide sequence, or at least one repeating unit of a sequence corresponding to at least one epitope of a selected GnRH molecule. The selected GnRH peptide sequences in the chimeras may all be the same, or may correspond to different derivatives, analogues, variants or epitopes of GnRH so long as they retain the ability to elicit an immune response. A detailed discussion of GnRH can be found in U.S. Pat. No. 4,975,420. Furthermore, a representative nucleotide sequence of a GnRH decapeptide is depicted in FIG. 1A. The subject GnRH sequence is modified by the substitution of a glutamine residue at the N-terminal in place of pyroglutamic acid which is found in the native sequence. This particular substitution provides a molecule that retains the native glutamic acid structure but also preserves the uncharged structure of pyroglutamate. Accordingly, the resulting peptide does not require cyclization of the glutamic acid residue and may be produced in the absence of conditions necessary to effect cyclization.

Because the GnRH sequence is relatively short, it can easily be generated using synthetic techniques. In leukotoxin-GnRH chimeras, a leukotoxin polypeptide sequence is used to confer immunogenicity upon associated GnRH polypeptides (as a carrier protein) to help elicit an adequate immune response toward endogenous GnRH in an immunized subject. Such immunization with GnRH can regulate fertility in a vaccinated subject by disruption of estrous cycles or spermatogenesis.

Particular leukotoxin-GnRH polypeptide chimeras used herein contain one or more GnRH portions having a plurality of selected GnRH polypeptide sequences. The GnRH portion of the chimera can comprise either multiple or tandem repeats of selected GnRH sequences, multiple or tandem repeats of selected GnRH epitopes, or any conceivable combination thereof. Suitable GnRH epitopes can be identified using routine techniques known in the art, or fragments of GnRH proteins may be tested for immunogenicity, and active fragments used in compositions in lieu of the entire polypeptide. When more than one GnRH multimer is included in the chimeric molecules, each GnRH portion can be the same or different from other included GnRH portions in the molecule.

The sequence of one particular GnRH multimer is depicted in FIG. 1B wherein four GnRH sequences, indicated at (1), (2), (3) and (4) respectively, are separated by triplet amino acid spacer sequences comprising various combinations of serine and glycine residues. In the subject multimer, every other GnRH sequence (e.g., those indicated at (2) and (4), respectively) contains a non-conservative amino acid substitution at the second position of the GnRH decapeptide comprising an Asp residue in place of the His residue found in the native GnRH sequence. The alternating GnRH multimeric sequence thus produced renders a highly immunogenic GnRH antigen. Other GnRH analogues corresponding to any single or multiple amino acid additions, substitutions and/or deletions can be used in either repetitive or alternating multimeric sequences. In one preferred leukotoxin-GnRH fusion, four copies of the GnRH portion depicted in FIG. 1B are fused to a leukotoxin molecule such that the leukotoxin molecule is flanked, on its N- and C-terminus, by two copies of the subject GnRH multimer.

The leukotoxin-GnRH immunogens can be produced recombinantly as a chimeric protein using the above-described methods. The nucleotide sequence coding for full-length P. haemolytica A1 leukotoxin has been determined. See, e.g., Lo, Infect. Immun. (1987) 55:1987-1996 and U.S. Pat. No. 5,055,400. Additionally, several variant leukotoxin gene sequences have been described in U.S. Pat. No. 5,476,657, International Publication No. WO 96/24675, published Aug. 15, 1996, and in commonly owned U.S. patent application Ser. No. 08/694,865, filed Aug. 9, 1996.

Similarly, the coding sequences for porcine, bovine and ovine GnRH have been determined (Murad et al. (1980) Hormones and Hormone Antagonists, in The Pharmacological Basis of Therapeutics, Sixth Edition), and the cDNA for human GnRH has been cloned so that its sequence has been well established (Seeburg et al. (1984) Nature 311:666-668). Additional GnRH polypeptides of known sequences have been disclosed, such as the GnRH molecule occurring in salmon and chickens (International Publication No. WO 86/07383, published Dec. 18, 1986). The GnRH coding sequence is highly conserved in vertebrates, particularly in mammals; and porcine, bovine, ovine and human GnRH sequences are identical to one another. The desired leukotoxin and GnRH genes can be cloned, isolated and ligated together using recombinant techniques generally known in the art. See, e.g., Sambrook et al., supra.

Particular examples of these GnRH immunogens are provided hereinbelow.

### Materials and Methods

Enzymes were purchased from commercial sources, and used according to the manufacturers' directions. Radionucleotides and nitrocellulose filters were also purchased from commercial sources.

In the cloning of DNA fragments, except where noted, all DNA manipulations were done according to standard procedures. See Sambrook et al., supra. Restriction enzymes, T₄ DNA ligase, E. coli, DNA polymerase I, Klenow fragment, and other biological reagents were purchased from commercial suppliers and used according to the manufacturers' directions. Double-stranded DNA fragments were separated on agarose gels.

cDNA and genomic libraries were prepared by standard techniques in pUC13 and the bacteriophage lambda gt11, respectively. See DNA CLONING: Vols I and II, supra.

P. haemolytica biotype A, serotype 1 (“A1”) strain B122 was isolated from the lung of a calf which died of pneumonic pasteurellosis and was stored at −70° C. in defibrinated blood. Routine propagation was carried out on blood agar plates or in brain heart infusion broth (Difco Laboratories, Detroit, Mich.) supplemented with 5% (v/v) horse serum (Gibco Canada Ltd., Burlington, Canada). All cultures were incubated at 37° C.

### EXAMPLE 1

### Construction of Leukotoxin-GnRH Chimeras

1. Isolation of P. haemolytica Leukotoxin Gene

To isolate the leukotoxin gene, gene libraries of P. haemolytica A1 (strain B122) were constructed using standard techniques. See, Lo et al., Infect. Immun., supra; DNA CLONING: Vols. I and II, supra; and Sambrook et al., supra. A genomic library was constructed in the plasmid vector pUC13 and a DNA library constructed in the bacteriophage lambda gt11. The resulting clones were used to transform E. coli and individual colonies were pooled and screened for reaction with serum from a calf which had survived a P. haemolytica infection and that had been boosted with a concentrated culture supernatant of P. haemolytica to increase anti-leukotoxin antibody levels. Positive colonies were screened for their ability to produce leukotoxin by incubating cell lysates with bovine neutrophils and subsequently measuring release of lactate dehydrogenase from the latter.

Several positive colonies were identified and these recombinants were analyzed by restriction endonuclease mapping. One clone appeared to be identical to a leukotoxin gene cloned previously. See, Lo et al., Infect. Immun., supra. To confirm this, smaller fragments were re-cloned and the restriction maps compared. It was determined that approximately 4 kilobase pairs of DNA had been cloned. Progressively larger clones were isolated by carrying out a chromosome walk (5′ to 3′ direction) in order to isolate full-length recombinants which were approximately 8 kb in length. The final construct was termed pAA114. This construct contained the entire leukotoxin gene sequence. lktA, a MaeI restriction endonuclease fragment from pAA114 which contained the entire leukotoxin gene, was treated with the Klenow fragment of DNA polymerase I plus nucleotide triphosphates and ligated into the SmaI site of the cloning vector pUC13. This plasmid was named pAA179. From this, two expression constructs were made in the ptac-based vector pGH432:lacI digested with SmaI. One, pAA342, consisted of the 5′-AhaIII fragment of the lktA gene while the other, pAA345, contained the entire MaeI fragment described above. The clone pAA342 expressed a truncated leukotoxin peptide at high levels while pAA345 expressed full length leukotoxin at very low levels. Therefore, the 3′ end of the lktA gene (StyI BamHI fragment from pAA345) was ligated to StyI BamHI-digested pAA342, yielding the plasmid pAA352. The P. haemolytica leukotoxin produced from the pAA352 construct is hereinafter referred to as LKT 352.

Several truncated versions of the leukotoxin gene were expressed from pAA114. These truncated forms were fusions with the B-galactosidase (lacZ) gene. Two fragments, LTX1.1 and LTX3.2, from an EcoRV Pst1 double digest, were isolated from pAA114 as purified restriction fragments (1.0 kb and 2.1 kb, respectively). These fragments were cloned into the cloning vector pTZ18R that had been digested with HincII and Pst1. The resulting vector, termed pLTX3P.1, was used to transform E. coli strain JM105. Transformed cells were identified by plating on media containing ampicillin plus Xgal and IPTG. Blue colonies indicated the presence of a functional lacZ gene. DNA from the transformed cells was analyzed by restriction endonuclease digestion and found to contain the 5′ end of the leukotoxin gene (lktC and lktA).

A leukotoxin EcoRV/Pst1 5′-fragment (from pLTX3P.1) was subcloned into the cloning vector pBR325 that had been digested with EcoR1 and Pst1. The pBR325 plasmid also contained the native leukotoxin promoter (obtained from pLTX3P.1) and a promoterless, full length lacZ gene. The resulting construct was used to transform E. coli JM105 and blue colonies were isolated from Xgal agar. The new construct was termed pAA101 (ATCC No. 67883). The P. haemolytica leukotoxin produced from the pAA101 construct is hereinafter referred to as “LKT 101.”

2. Construction of LKT-GnRH Fusions

Representative LKT-GnRH fusions were constructed as follows. Oligonucleotides containing sequences corresponding to single copy GnRH and GnRH as four multiple repeats were constructed on a Pharmacia Gene Assembler using standard phosphoramidite chemistry. The sequences of these oligonucleotides are shown in FIGS. 1A and 1B. The subject oligonucleotides were annealed and ligated into the vector pAA352 (ATCC No. 68283, and described above), which had been digested with the restriction endonuclease BamH1. This vector contains the P. haemolytica leukotoxin gene. The ligated DNA was used to transform E. coli strain MH3000. Transformants containing the oligonucleotide inserts were identified by restriction endonuclease mapping.

An eight copy GnRH tandem repeat sequence was prepared by annealing the four copy GnRH oligonucleotides and ligating them into a vector which had been digested with the restriction endonuclease BamH1. The oligomers were designed to disable the upstream BamH1 site when inserted and to ensure that the insertion of additional copies of the oligomer would be oriented in the proper reading frame. The sequence of the subject oligonucleotide is shown in FIG. 1B. Plasmid DNA from the E. coli MH3000 strain was then isolated and used to transform the strain JM105. The recombinant plasmids were designated pCB113 (LKT 352:4 copy GnRH, ATCC Accession No. 69749) and pCB112 (LKT 352:8 copy GnRH).

3. Construction of Shortened LKT Carrier Peptide

A shortened version of the recombinant leukotoxin peptide was constructed from the recombinant gene present on the plasmid pAA352 (as described above). The shortened LKT gene was produced by deleting an internal DNA fragment of approximately 1300 bp in length from the recombinant LKT gene as follows.

The plasmid pCB113, (ATCC Accession No. 69749) which includes the LKT 352 polypeptide fused to four copies of the GnRH polypeptide, was digested with the restriction enzyme BstB1 (New England Biolabs). The resultant linearized plasmid was then digested with mung-bean nuclease (Pharmacia) to remove the single stranded protruding termini produced by the BstB1 digestion. The blunted DNA was then digested with the restriction enzyme Nae1 (New England Biolabs), and the digested DNA was loaded onto a 1% agarose gel where the DNA fragments were separated by electrophoresis. A large DNA fragment of approximately 6190 bp was isolated and purified from the agarose gel using a Gene Clean kit (Bio 101), and the purified fragment was allowed to ligate to itself using bacteriophage T4 DNA ligase (Pharmacia). The resulting ligation mix was used to transform competent E. coli JM105 cells, and positive clones were identified by their ability to produce an aggregate protein having a molecular weight of approximately 57 KDa. The recombinant plasmid thus formed was designated pCB111, (ATCC Accession No. 69748), and produces a shortened leukotoxin polypeptide (hereinafter referred to as “LKT 111”) fused to four copies of GnRH polypeptide. Plasmid pCB114 is identical to pCB111 except that the multiple copy GnRH sequence (corresponding to the oligomer of FIG. 1B) was inserted twice.

4. Construction of an LKT-GnRH Fusion Having 8 Copy Amino Terminal and Carboxyl Terminal GnRH Multimers

A recombinant LKT-GnRH fusion molecule having two 8 copy GnRH multimers, one arranged at the N′-terminus of LKT 111 and the other arranged at the C′-terminus of LKT 111, was constructed from the LKT-GnRH fusion sequence obtained from the pCB114 plasmid by ligating the multiple copy GnRH sequence (corresponding to the oligomer of FIG. 1B) twice at the 5′ end of the LKT 111 coding sequence. A synthetic nucleic acid molecule having the following nucleotide sequence: 5′-ATGGCTACTGTTATAGATCGATCT-3′ (SEQ ID NO.:______) was ligated at the 5′ end of the multiple copy GnRH sequences. The synthetic nucleic acid molecule encodes an eight amino acid sequence, Met-Ala-Thr-Val-Ile-Asp-Arg-Ser (SEQ ID NO.:______). The resulting recombinant molecule thus contains in the order given in the 5′ to 3′ direction: the synthetic nucleic acid molecule; a nucleotide sequence encoding a first 8 copy GnRH multimer; a nucleotide sequence encoding the shortened LKT peptide (LKT 111); and a nucleotide sequence encoding a second 8 copy GnRH multimer.

The recombinant molecule was circularized, and the resulting molecule was used to transform competent E. coli JM105 cells. Positive clones were identified by their ability to produce an aggregate protein having a molecular weight of approximately 74 KDa. The recombinant plasmid thus formed was designated pCB122 which produces the LKT 111 polypeptide fused to 16 copies of GnRH polypeptide.

A series of recombinant LKT-GnRH fusion molecules were then derived from pCB122 as follows. The 8 copy GnRH multimer at the 5′ end of the pCB122 construct was amplified using PCR. The copied GnRH multimer sequence was then modified to provide a GnRH insert that could be ligated into the Nsi1 site of the leukotoxin carrier in pCB122 and maintain the reading frame. Synthetic sequences, encoding additional amino acids flanking the GnRH insert, were also ligated to the insert. The flanking amino acids were required to successfully use PCR to copy the GnRH insert and to link the insert to the leukotoxin molecule. The resulting construct, termed pCB133, contained an additional 8 copies of GnRH that were inserted into the Nsi1 site of the shortened LKT peptide (LKT 111) in the pCB122 construct.

A further construct, termed pCB134, was constructed in the same manner as pCB133, however, the 8 copy GnRH insert was inserted into the Stu1 site of the LKT 111 carrier in the pCB122 construct. A set of flanking synthetic sequences (different than the ones used in the construction of the pCB133 construct) were added to the GnRH insert in order to link it to LKT 111. pCB134 thus contains an additional 8 copies of GnRH that are inserted into the Stu1 site of the shortened LKT peptide (LKT 111) in the pCB122 construct.

The Nsi1 insert from pCB133, containing the 8 copy GnRH insert described above, was excised and ligated into the Nsi1 site in pCB134 to provide a further construct termed pCB135. The pCB135 construct produced a chimeric molecule comprising the LKT 111 polypeptide fused to GnRH multimers (8 copies each) at 4 different locations, for a total 32 copies of GnRH in the molecule.

A further construct, termed pCB136, was derived from pCB122 by inserting into the Stu1 site of the LKT 111 sequence a synthetic polynucleotide encoding a number of “universal T-cell epitope” peptide sequences interspersed between GnRH sequences. Universal T-cell epitopes appear to stimulate T-cell immune responses in all species tested. See e.g. Sinigaglia et al. (1988) Nature 336:778-780; Panina-Bordignon et al. (1989) Eur. J. Immunol. 19:2237-2242; O'Sullivan et al. (1990) J.

Immun. 145:1799-1808; and O'Sullivan et al. (1991) J. Immun. 147:2663-2669. In particular, the polynucleotide insert included, in the 5′ to 3′ direction, a sequence coding for the universal T-cell epitope from tetanus toxin, a GnRH sequence, a sequence coding for the T-cell epitope from diphtheria toxin, a GnRH sequence, a sequence coding for the T-cell epitope from sperm whale myoglobin, and a final GnRH sequence. Each GnRH sequence was separated from adjacent T-cell epitopes by 2 lysine residues which serve as the site of action for the enzyme cathepsin. Cathepsin is a protease that is involved in the degradation of antigens for presentation to the immune system.

5. Purification of LKT-antigen Fusions

The recombinant LKT-GnRH fusions were purified using the following procedure. For each fusion, five to ten colonies of the transformed E. coli strains were inoculated into 10 mL of TB broth supplemented with 100 micrograms/mL of ampicillin and incubated at 37° C. for 6 hours on a G10 shaker, 220 rpm. Four mL of this culture was diluted into each of two baffled Fernbach flasks containing 400 mL of TB broth+ampicillin and incubated overnight as described above. Cells were harvested by centrifugation for 10 minutes at 4,000 rpm in polypropylene bottles, 500 mL volume, using a Sorvall GS3 rotor. The pellet was resuspended in an equal volume of TB broth containing ampicillin which had been prewarmed to 37° C. (i.e., 2×400 ml), and the cells were incubated for 2 hours as described above. 3.2 mL of isopropyl-B,D-thiogalactopyranoside (IPTG, Gibco/BRL), 500 mM in water (final concentration=4 mM), was added to each culture in order to induce synthesis of the recombinant fusion proteins. Cultures were incubated for two hours. Cells were harvested by centrifugation as described above, resuspended in 30 mL of 50 mM Tris-hydrochloride, 25% (w/v) sucrose, pH 8.0, and frozen at −70° C. The frozen cells were thawed at room temperature after 60 minutes at −70° C., and 5 mL of lysozyme (Sigma, 20 mg/mL in 250 mM Tris-HCl, pH 8.0) was added. The mixture was vortexed at high speed for 10 seconds and then placed on ice for 15 minutes. The cells were then added to 500 mL of lysis buffer in a 1000 mL beaker and mixed by stirring with a 2 mL pipette. The beaker containing the lysed cell suspension was placed on ice and sonicated for a total of 2.5 minutes (5-30 second bursts with 1 minute cooling between each) with a Braun sonicator, large probe, set at 100 watts power. Equal volumes of the solution were placed in Teflon SS34 centrifuge tubes and centrifuged for 20 minutes at 10,000 rpm in a Sorvall SS34 rotor. The pellets were resuspended in a total of 100 mL of sterile double distilled water by vortexing at high speed, and the centrifugation step repeated. Supernatants were discarded and the pellets combined in 20 mL of 10 mM Tris-HCl, 150 mM NaCl, pH 8.0 (Tris-buffered saline) and the suspension frozen overnight at −20° C.

The recombinant suspension was thawed at room temperature and added to 100 mL of 8 M Guanidine HCl (Sigma) in Tris-buffered saline and mixed vigorously. A magnetic stir bar was placed in the bottle and the solubilized sample was mixed at room temperature for 30 minutes. The solution was transferred to a 2000 mL Erlenmeyer flask and 1200 mL of Tris-buffered saline was added quickly. This mixture was stirred at room temperature for an additional 2 hours. 500 mL aliquots were placed in dialysis bags (Spectrum, 63.7 mm diameter, 6,000-8,000 MW cutoff, #132670, from Fisher scientific) and these were placed in 4,000 mL beakers containing 3,500 mL of Tris-buffered saline +0.5 M Guanidine HCl. The beakers were placed in a 4° C. room on a magnetic stirrer overnight after which dialysis buffer was replaced with Tris-buffered saline+0.1 M Guanidine HCl and dialysis continued for 12 hours. The buffer was then replaced with Tris-buffered saline+0.05 M Guanidine HCl and dialysis continued overnight. The buffer was replaced with Tris-buffered saline (no guanidine), and dialysis continued for 12 hours. This was repeated three more times. The final solution was poured into a 2000 mL plastic roller bottle (Corning) and 13 mL of 100 mM PMSF (in ethanol) was added to inhibit protease activity. The solution was stored at −20° C. in 100 mL aliquots.

To confirm that the fusion proteins had been isolated, aliquots of each preparation were diluted 20-fold in double distilled water, mixed with an equal volume of SDS-PAGE sample buffer, placed in a boiling water bath for five minutes and run through 12% polyacrylamide gels. Recombinant leukotoxin controls were also run. All fusion proteins were expressed at high levels as inclusion bodies.

### EXAMPLE 2

### Administration of GnRH Immunogens

The following study was carried out to compare the efficacy and uniformity of vaccination with GnRH immunogens administered either to the ear or intramuscularly into the neck of porcine subjects. Five different GnRH immunogens were used in the trial, particularly the leukotoxin-GnRH chimeras obtained from the pCB122, pCB133, pCB134, pCB135 and pCB136 constructs described above in Example 1.

All of the recombinant GnRH immunogens were produced in E. coli, and were combined with the VSA-3 oil-in-water adjuvant (manufactured by MVP Laboratories, Ralston, Nebr.). The vaccines were formulated to deliver 40 μg of the GnRH immunogen in a volume of 1.0 mL when given by conventional needle and syringe, or 0.5 mL when administered with a jet injector.

Needle injections were given intramuscularly, 8 to 10 cm behind the ear and 6 to 10 cm on either side of the midline, using a 2 mL syringe and a 1 inch, 20 gauge needle. The needleless injections (jet injections) were administered with the Bioject 2000 injection system, (manufactured by Bioject, Portland, Oreg.). The jet injector was fitted with a specialized 1 mL syringe having an orifice size which allowed the jet of liquid to pierce the skin, and be deposited at a subcutaneous location in the ear. These ear injections were given into the outer surface of the pinna, and accomplished by grasping the tip of the ear to immobilize it and create a flat surface which was capable of resisting the pressure of the injection device as it was held on the surface of the ear. The vaccine penetrated the skin and moved laterally in a thin sheet along the surface of the inner cartilaginous structure. The animal subjects tolerated this procedure with little or no evidence of pain or distress.

Each of the five vaccine formulations were administered intramuscularly to 10 animals by needle injection into the neck, and subcutaneously to 5 different animals by jet injection to the ear. More particularly, the animals were injected to the left ear or neck at 21 days of age, and to the right ear or neck 35 days later. Animals were observed twice weekly to evaluate injection site reactions. Blood samples were collected at the time of the booster injection, and 14 and 28 days later. Serum was assayed for GnRH antibodies by a standard procedure.

Particularly, serum samples were taken, appropriate dilutions were made in buffer, and aliquots were placed into test tubes. A standard amount of iodinated (¹²⁵I) GnRH was then added to each tube. The final serum dilution assayed were 1:5,000 and 1:20,000. After incubation for 48 hours at 5° C., a 1 mL aliquot of 1% charcoal suspension in buffer was added. The tubes were centrifuged to sediment the charcoal, and radioactivity of the tubes containing the pellets was counted. In this method, the charcoal adsorbs the ¹²⁵I-labeled GnRH, and a calculation can be made to determine the amount of ¹²⁵I-GnRH bound to antibody in the sample. The results are expressed below in Table 1 as % of the added ¹²⁵I-GnRH that bound to the sample. Higher values indicate higher antibody titres.

TABLE 1
Antibody Titres at 1:5000 Dilution (Means ± SF)
with Five Different Antigens and Two Methods of Injection

| Site/Method | | | | | | |
|---|---|---|---|---|---|---|
| of Injection | n | pCB122 | pCB133 | pCB134 | pCB135 | pCB136 |
| Needle-Neck | 10 | 47.7 ± 9.4 | 33.4 ± 7.3 | 31.1 ± 7.6 | 30.2 ± 7.3 | 32.8 ± 8.7 |
| Jet-Ear | 5 | 51.3 ± 7.8 | 47.8 ± 13.5 | 55.2 ± 7.8 | 42.0 ± 14.2 | 61.1 ± 9.4 |

The mean antibody titres with the two different methods of injection are depicted below in Table 2.

TABLE 2
Mean Antibody Titres with Two
Different Methods or Routes of Injection

| | Mean ± SE | n |
|---|---|---|
| Needle-Neck | 35.0 ± 3.6^{a} | 50 |
| Jet-Ear | 51.5 ± 4.7^{b} | 25 |

a vs b, p < .05

The serological evidence depicted in Tables 1 and 2 shows a better response to all five GnRH immunogens when delivered to the ear by jet injector. These results clearly demonstrate that the ear is a preferred site for immunization with the GnRH immunogens, providing a superior antibody titre as compared with the immunizations delivered via intramuscular injection in the neck. The ear also provides a preferred site for vaccine delivery since the tissue of the pinna is generally uniform from animal to animal, allowing the vaccine to be presented in a consistent fashion.

### EXAMPLE 3

### Administration of Vaccine Compositions to the Ear

The following study was carried out to compare the efficacy of GnRH vaccination carried out via subcutaneous injection into the neck, or intradermal delivery into the ear.

More particularly, two groups of 20 pigs each (10 male and 10 female) were injected either subcutaneously in the neck with 0.2 mL of vaccine containing 40 μg of the leukotoxin-GnRH chimera obtained from the pCB122 construct (Example 1), or 0.2 mL of the same vaccine intradermally in the ear. The vaccine compositions contained VSA-3 adjuvant (Example 2), and were delivered to the neck or ear via needle and syringe. The primary injection was given at 21 days of age and the booster dose was administered 35 days later. Blood was collected 14 and 28 days after the boost and analyzed for anti-GnRH antibodies as described above in Example 2. Antibody titres were then expressed as % binding of ¹²⁵-GnRH in serum diluted at 1:5000. These results are reported below in Table 3.

TABLE 3

Antibody Titres after the Booster

| Site of Injection | Day 14 | Day 28 |
|---|---|---|
| Neck | 60 ± 4.3* | 56 ± 4.9 |
| Ear | 66 ± 3.6 | 65 ± 5.0 |

*Mean values ± standard errors

As can be seen, antibody titers were highest when the vaccine was given in the ear, and these titres remained high for the duration of the trail (28 days after the booster immunization). These data confirm the usefulness of the ear as a vaccination site.

### EXAMPLE 4

### Administration of Vaccine Compositions to the Ear

In order to further assess the efficacy of the vaccination methods which target the ear, the following study was carried out. In this study one experimental groups consisting of 20 pigs received either 0.2 or 0.4 mL of a vaccine composition containing a water-in-oil adjuvant (Seppic ISA-70, available from Seppic, Inc., Castres, France). The vaccine composition included 40 μg of the leukotoxin-GnRH chimera obtained from the pCB122 construct (see Example 2) and was given subcutaneously in the ear as a single dose to 60 day-old pigs. Table 4 reports the anti-GnRH antibody titres (% binding of ¹²⁵I-GnRH at a 1:5000 dilution) obtained from these animals at days 14, 28, 42 and 56 post injection.

TABLE 4

| | Day After Injection | | | |
|---|---|---|---|---|
| Volume | 14 | 28 | 42 | 56 |
| 0.2 ml | 2.5 ± 0.8* | 14.5 ± 3.2 | 22.5 ± 3.7 | 34.6 ± 4.4 |
| 0.4 ml | 2.2 ± 0.6 | 19.1 ± 3.6 | 32.9 ± 4.8 | 49.2 ± .50 |

*Mean values ± standard errors

As can be seen by the results reported in Table 4, a single ear injection using a water-in-oil adjuvanted-vaccine composition evoked a strong antibody response which was still increasing 56 days after the single injection. These data indicate that the ear is a site which responds well to different classes of adjuvants. Targeting such vaccine compositions to the ear provides advantages with respect to tissue residue, ease of administration, and safety of animal technicians when administering potentially hazardous vaccines.

### EXAMPLE 5

### Comparison of Adjuvant Systems, Booster Vaccinations

The following study was carried out in order to assess the efficacy of targeting the mammalian ear for booster vaccinations. Leukotoxin-GnRH chimeras obtained from the pCB122 construct (Example 1) were administered to cattle using either an oil-in-water adjuvant, or a water-in-oil adjuvant. More particularly, all of the cattle used in the study were primed by vaccination with 200 μg of the pCB122 chimera immunogen combined with a suitable adjuvant (a water-in-oil emulsion formed with a metabolizable oil (Squalene)) to provide a final volume of 2.0 mL. The prime was carried out using needle and syringe to deliver the vaccine composition im into the neck. For the booster immunization, three experimental groups of cattle were established by boosting with the following vaccines: (Group 1) received 200 μg of the pCB122 chimera immunogen in a 2.0 mL volume of an oil-in-water adjuvant (VSA3), administrations were carried out via subcutaneous injection to the neck using a standard needle and syringe; (Group 2) received 200 μg of the pCB122 chimera immunogen in a 0.5 mL volume of the VSA3 adjuvant, administrations were carried out via subcutaneous injection to the ear carried out using a jet injection device; and (Group 3) received 300 μg of the pCB122 chimera immunogen in a water-in-oil adjuvant (Seppic ISA-70), administrations were carried out via subcutaneous injection to the ear via jet injection device.

Table 5, below, provides the anti-GnRH antibody titres from each group of animals (reported as % binding of ¹²⁵I-GnRH at a 1:100 dilution) at day 21 and day 105 post booster vaccination.

TABLE 5

| | Day 21 | Day 105 |
|---|---|---|
| (Group 1) Neck, | 76.5 ± 2.32* | 65.0 ± 6.21 |
| VSA3 Adjuvant | | |
| (Group 2) Ear, | 67.7 ± 4.53 | 53.1 ± 3.56 |
| VSA3 Adjuvant | | |
| (Group 3) Ear, | 72.2 ± 4.69 | 69.5 ± 7.5 |
| W/O Adjuvant | | |

*Group mean ± standard error of mean

As can be seen by the data reported in Table 5, a booster vaccination administered to the ear (with either adjuvant formulation) provided an equivalent antibody response to the subcutaneous booster vaccination that was administered to the neck.

### EXAMPLE 6

### Single-Dose Vaccination to Mammalian Ear

In yet a further study, 29 heifers were vaccinated once in the ear subcutaneously via jet injector device, using 200 μg of the leukotoxin-GnRH chimera obtained from the pCB122 construct. The vaccine was formulated using a water-in-oil adjuvant (Seppic ISA-70). The anti-GnRH antibody titres for these heifers (% binding of ¹²⁵1I-GnRH at a 1:100 dilution) at days 0, 21, and 35 post vaccination are reported below in Table 6.

TABLE 6

| Day 0 | Day 21 | Day 35 |
|---|---|---|
| 2.86 ± 0.57* | 16.47 ± 3.06 | 21.41 ± 3.98 |

*Group mean ± standard error of the mean

These data demonstrate that a single dose of a GnRH vaccine composition administered to the ear provides a substantial primary vaccine response at Day 35. Thus, the ear is an effective vaccination site for both primary and booster vaccinations in cattle.

Thus, methods for immunizing a mammalian subject against an endogenous immunogen via administration of a vaccine composition to the ear have been disclosed. Although preferred embodiments of the subject invention have been described in some detail, it is understood that obvious variations can be made without departing from the spirit and the scope of the invention as defined by the appended claims.

### DEPOSITS OF STRAINS USEFUL IN PRACTICING THE INVENTION

A deposit of biologically pure cultures of the following strains was made with the American Type Culture Collection (ATCC), 12301 Parklawn Drive, Rockville, Md. The accession number indicated was assigned after successful viability testing, and the requisite fees were paid. The deposits were made under the provisions of the Budapest Treaty on the International Recognition of the Deposit of Microorganisms for the Purpose of Patent Procedure and the Regulations thereunder (Budapest Treaty). This assures maintenance of viable cultures for a period of thirty (30) years from the date of deposit and at least five (5) years after the most recent request for the furnishing of a sample of the deposit by the depository. The organisms will be made available by the ATCC under the terms of the Budapest Treaty, which assures permanent and unrestricted availability of the cultures to one determined by the U.S. Commissioner of Patents and Trademarks to be entitled thereto according to 35 U.S.C. §122 and the Commissioner's rules pursuant thereto (including 37 C.F.R. §1.12). Upon the granting of a patent, all restrictions on the availability to the public of the deposited cultures will be irrevocably removed.

These deposits are provided merely as convenience to those of skill in the art, and are not an admission that a deposit is required under 35 U.S.C. §112. The nucleic acid sequences of these plasmids, as well as the amino acid sequences of the polypeptides encoded thereby, are incorporated herein by reference and are controlling in the event of any conflict with the description herein. A license may be required to make, use, or sell the deposited materials, and no such license is hereby granted.

| Strain | Deposit Date | ATCC No. |
|---|---|---|
| P. haemolytica serotype 1 B122 | February 1, 1989 | 53863 |
| pAA101 in E. coli JM105 | February 1, 1989 | 67883 |
| pAA352 in E. coli W1485 | March 30, 1990 | 68283 |
| pCB113 in E. coli JM105 | February 1, 1995 | 69749 |
| pCB111 in E. coli JM105 | February 1, 1995 | 69748 |

We claim:

1. A method for inducing an immune response against an endogenous molecule in a mammalian subject, comprising administering to the ear of said subject an effective amount of a vaccine composition comprising an immunogen derived from said molecule, and a pharmaceutically acceptable vehicle, wherein said vaccine composition is capable of inducing an immune response against said molecule.

2. The method of claim 1, wherein the endogenous molecule is a hormone.

3. The method of claim 2, wherein the hormone is GnRH.

4. The method of claim 1, wherein the endogenous molecule is a hormone receptor.

5. The method of claim 1, wherein the vaccine composition is administered to the subject via subcutaneous delivery into the pinna of said subject's ear.

6. The method of claim 1, wherein the vaccine composition is administered to the subject via intradermal delivery into the pinna of said subject's ear.

7. The method of claim 1, wherein the vaccine composition is administered in more than one dose.

8. The method of claim 1, wherein the vaccine composition comprises a polypeptide immunogen linked to a carrier molecule.

9. The method of claim 1, wherein the vaccine composition further comprises an adjuvant.

10. The method of claim 9, wherein the adjuvant is an oil-in-water formulation.

11. The method of claim 1, wherein the vaccine composition is administered with a needleless or jet injector device.

12. The method of claim 1, wherein the vaccine composition is administered in solid form.

13. The method of claim 12, wherein the vaccine composition is in solid, particulate form.

14. The method of claim 12, wherein the vaccine composition is administered as a solid dose implant.

15. The method of claim 1, wherein the vaccine composition comprises a nucleic acid molecule which encodes said immunogen.

16. The method of claim 1, wherein the mammalian subject is bovine.

17. The method of claim 1, wherein the mammalian subject is porcine.

18. A method for delivering a selected endogenous immunogen to a mammalian subject, comprising administering to the ear of said subject an effective amount of a vaccine composition comprising the endogenous immunogen and a pharmaceutically acceptable vehicle.
